(19) United States
(12) Patent Application Publication
(10) Pub. No.: US 2012/0253156 A1
(43) Pub. Date: Oct. 4, 2012

(54) METHOD AND APPARATUS FOR PROCESSING PHOTOPLETHYMOGRAPH SIGNALS

(75) Inventor: Jens Muhlsteff, Aachen (DE)

(73) Assignee: KONINKLIJKE PHILIPS ELECTRONICS N.V., EINDHOVEN (NL)

(21) Appl. No.: 13/513,912

(22) PCT Filed: Nov. 24, 2010
(86) PCT No.: PCT/IB10/55397

§ 371 Date: Jun. 5, 2012

(30) Foreign Application Priority Data
Dec. 21, 2009 (EP) 09180067.2

(51) Int. Cl. A61B 5/024; A61B 5/1455

(52) U.S. Cl. 600/324

(57) ABSTRACT

The disclosure relates to the field a method of and apparatus for processing a photoplethysmograph signal to support the analysis of photoplethysmograph signals in clinical scenarios. A derivative of a photoplethysmograph signal acquired over a time period is calculated. The derivative of the acquired photoplethysmograph signal with respect to time is analyzed and displayed in an x-y diagram as a function of the acquired photoplethysmograph signal or vice versa.

### FIELD OF THE INVENTION

The invention relates to a method of and apparatus for processing photoplethysmograph signals to support the analysis of photoplethysmograph signals in clinical scenarios.

### BACKGROUND OF THE INVENTION

Besides an electrocardiogram (ECG), a photoplethysmograph (PPG) signal is one of the most often acquired signals in clinical scenarios such as in anesthesia or intensive care. A PPG signal can be measured continuously and comfortably from the finger, ear or forehead of a subject, i.e. a patient. A PPG is often obtained by using a pulse oximeter which illuminates the skin and measures changes in light absorption. A conventional pulse oximeter monitors the perfusion of blood to the dermis and subcutaneous tissue of the skin.

Normally, from a PPG signal the heart rate and the Sp02 of a patient are estimated. However, not all information embedded in the PPG waveform and its morphology is used in the analysis of the PPG signal. For example the PPG waveform provides additional information on the cardio-vascular status of a subject which could be tracked over time to assist in an early detection of cardio-vascular responses or changes of a subject.

However, in clinical practice a physician is not able to track and compare PPG waveforms and morphologies in an easy and intuitive way for a specific patient during a monitoring period. Lacking is a simple and, for a physician, intuitive concept to interpret

PPG pulse waveforms that are related to clinical contexts like for example drug responses and disease progression, in an easy way.

### SUMMARY OF THE INVENTION

It is an object of the invention to provide a method and apparatus for an easy and intuitive analysis of a PPG signal which is more robust and assists a physician in the interpretation of a PPG signal and enables a correlation of the PPG waveform with a related clinical context, for example to a cardio-vascular state of a patient.

With respect to the method, this object is achieved by a method of processing a photoplethysmograph signal retrieved from a subject, said method comprising the steps of:

acquiring the photoplethysmograph signal over a time period;

calculating a derivative of the acquired photoplethysmograph signal; and

analyzing the derivative of the acquired photoplethysmograph signal with respect to time as a function of the acquired photoplethysmograph signal or vice versa.

With the method according to the invention, an easy and intuitive way to analyze PPG waveforms and morphologies is provided, the results of which may be presented, for example, on a patient monitor during monitoring periods or diagnostic procedures. The derivative of the PPG signal with respect to time as a function of the PPG signal itself or, vice versa, the PPG signal as a function the derivative of the PPG signal with respect to time provides an additional and an improved way of recognizing and indicating specific PPG waveforms or parts of PPG waveforms. The analysis of this function, whether done visually via an x-y graph or automatically by a processor, further assists the physician in the interpretation of the PPG signal and enables the physician to relate the PPG signal to a specific clinical context. The analysis of this function provides for an easy interpretation of changes of the PPG waveforms over time, like for example PPG amplitudes and amplitude changes, systolic and diastolic slopes, oscillations. The analysis of this function further provides for a much faster and more robust recognition of, for example, the dicrotic notch, and a more robust discrimination of PPG waveform changes in systolic and diastolic phase.

The analysis of this function reduces the chances of misinterpreting the PPG signals, because this function provides an improved distinction between, for example, PPG signals acquired from different postures of the subject thereby ensuring that only PPG signals acquired for the same posture of the subject are compared. Furthermore, an earlier detection of critical states of a patient is enabled, for example, due to vasodilatation and/or vasoconstriction and the chance on misinterpretation of the PPG signal is reduced because of the more robust analysis of the derivative of the PPG signal as a function of the PPG signal. The analysis of the PPG signal becomes even more robust if the conventional PPG waveform, i.e. the PPG signal as a function of time, is additionally used in the analysis. Furthermore, specific features or parts of this function may be characterized by one or more parameters, such as for example the dicrotic notch. By outputting these parameters as result of the analysis, the invention thereby further assists the physician in analyzing and monitoring of the patient via the PPG signal.

In an embodiment, the proposed method can be adapted to specific application scenarios. In particular, the method can be adapted for a specific application by, for example, the use of a first or higher derivative of the PPG signal and/or different pre-processing steps of the PPG signal, like for example amplitude normalization, artifact rejection and/or high- and low pass filtering.

This object is also achieved by a photoplethysmograph measurement apparatus comprising a sensor for acquiring a photoplethysmograph signal corresponding to a property of blood in the subject tissue, and a processor connected to the sensor and adapted to receive and process the photoplethysmograph signal from the sensor. The processor is adapted to calculate a derivative with respect to time of the photoplethysmograph signal received from the sensor, and to analyze the derivative of the photoplethysmograph signal as a function of the photoplethysmograph signal or vice versa.

This object is also achieved by a patient monitoring system comprising the photoplethysmograph measurement apparatus according to the invention.

This object is also achieved by a computer program for instructing a computer to perform the method according to the invention. This object is also achieved by a computer-readable medium such as a storage device, such as a floppy disk, CD, DVD, Blue Ray disk, or a random access memory (RAM), containing a set of instructions that causes a computer to perform a method according to the invention.

Advantageous embodiments are defined by the dependent claims.

### BRIEF DESCRIPTION OF THE DRAWINGS

These and other aspects of the invention will be apparent from and elucidated with reference to the embodiments described hereinafter. In the drawings: FIGS. 1a, 1b and 1c show a PPG signal acquired during a head up tilt table test (HUTT);

FIG. 2 is a chart showing PPG signals acquired from a subject during a sequence of posture changes;

FIGS. 3a, 3b and 3c depict x-y diagrams of a PPG signal according to an aspect of the invention;

FIGS. 4a and 4b depict a further x-y diagram of a PPG signal according to an aspect of the invention;

FIG. 5 shows an x-y diagram of a PPG signal according to an aspect of the invention comparing different states of a patient;

FIG. 6 depicts an x-y diagram of a PPG signal according to an aspect of the invention, when the subject changes posture with a state-of-the art photoplethysmograph measurement apparatus;

FIG. 7 depicts a further x-y diagram of a PPG signal according to a further aspect of the invention, when the posture of the subject is taken into account;

FIG. 8 shows a schematic plot of an embodiment of a photoplethysmograph measurement apparatus according to the invention;

FIG. 9 shows a schematic plot of a further embodiment of a photoplethysmograph measurement apparatus according to the invention;

FIG. 10 shows a schematic plot of a further embodiment of a photoplethysmograph measurement apparatus according to the invention; and

FIG. 11 shows an x-y diagram of a PPG signal of a basal PPG according to an aspect of the invention.

### DETAILED DESCRIPTION OF EMBODIMENTS

A photoplethysmograph (PPG) is an optically obtained plethysmograph, which is a volumetric measurement of an organ. It can be obtained by a pulse oximeter which illuminates the skin and measures changes in light absorption. A conventional pulse oximeter monitors the perfusion of blood to the dermis and subcutaneous tissue of the skin. Besides the ECG, the PPG signal is one of the most often acquired signals in clinics, especially in anesthesia or intensive care. Typically, the PPG is measured from the finger, ear or forehead. From this PPG signal the heart rate and the patient's SpO2 can be estimated. However, while currently only the heart rate and the patient's SpO2 are estimated routinely from the PPG signal, the PPG waveform provides additional information on a subject cardio-vascular state for detection of, for example, cardio-vascular responses of a subject during interventions.

As an example, the upper diagram a) of FIG. 1 shows the PPG morphology change during a head up tilt table test (HUTT). This test involves the patient being tilted, always with the head-up, at different angles for a period of time. The upper diagram a) of FIG. 1 shows the PPG signal 22 as a function of time and the block shaped curve 21 visualizes when the patient is tilted. The lower left diagram b) of FIG. 1 shows an enlarged view of the

PPG signal 22 and shape before a nitro-glycerin administration and the diagram c) on the lower right side of FIG. 1 shows an enlarged view of the PPG signal 22 and shape after a nitro-glycerin administration. In this case, an increase of the PPG pulse amplitude as well as a change of the relative height of the maximum PPG peak and the secondary peak in the PPG pulse wave, also called the dicrotic notch, is clearly visible, indicating a significant change of the cardio-vascular status of the patient due to the dilatation effect of the administered nitro-glycerin. However, from this diagram it is not easy for a physician to interpret the PPG waveform and, hence, it is not straightforward and simple to relate the PGG signal 22 to an appropriate clinical context, which makes this diagram, the PPG signal 22 as a function of time, not suitable for a clinical daily routine analysis. This is one of the reasons, why the analysis of the PPG morphology, or waveform, is still not accepted by clinicians. In clinical practice a physician is not able to track, analyze and compare PPG morphologies and waveforms easily and intuitively for a specific patient during a monitoring period. The information on, for example, the cardio-vascular status of a patient that is embedded in the

PPG waveform is typically not used since:

there is no intuitive visualization concept of PPG morphologies that can be related to a specific clinical context or patient status;

the shape of the PPG waveforms is context sensitive, for example due to posture change, physical activities and/or hydrostatic effects, which makes the interpretation and analysis of the PPG waveform difficult;

PPG signals acquired at different moments in time are normally not stored for comparison reasons;

the interpretation of PPG signal changes in different phases of a pulse, for example systolic versus diastolic, is difficult; and/or

PPG signals belonging to different heart rates cannot be normalized in time easily without significant signal distortion.

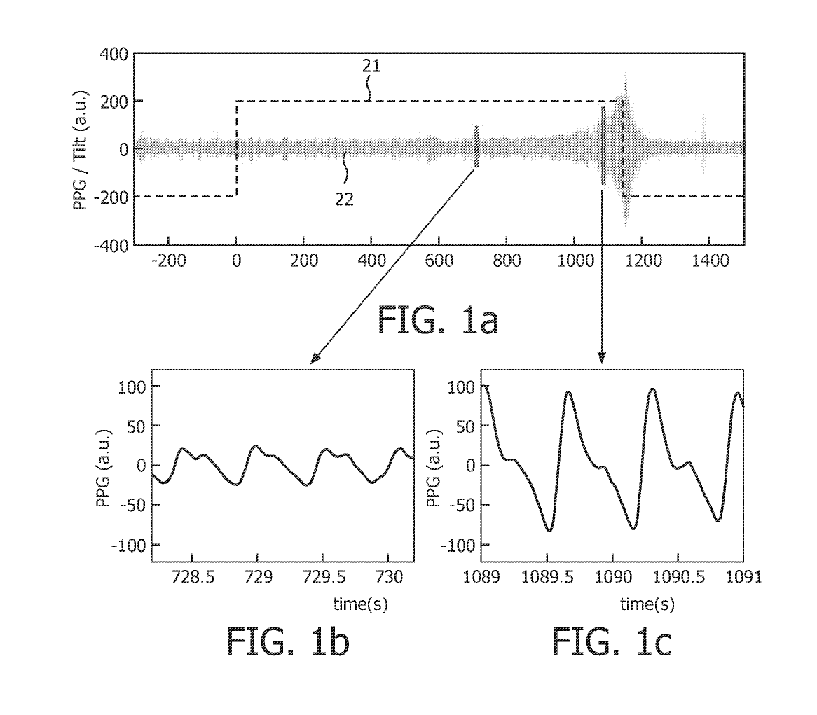

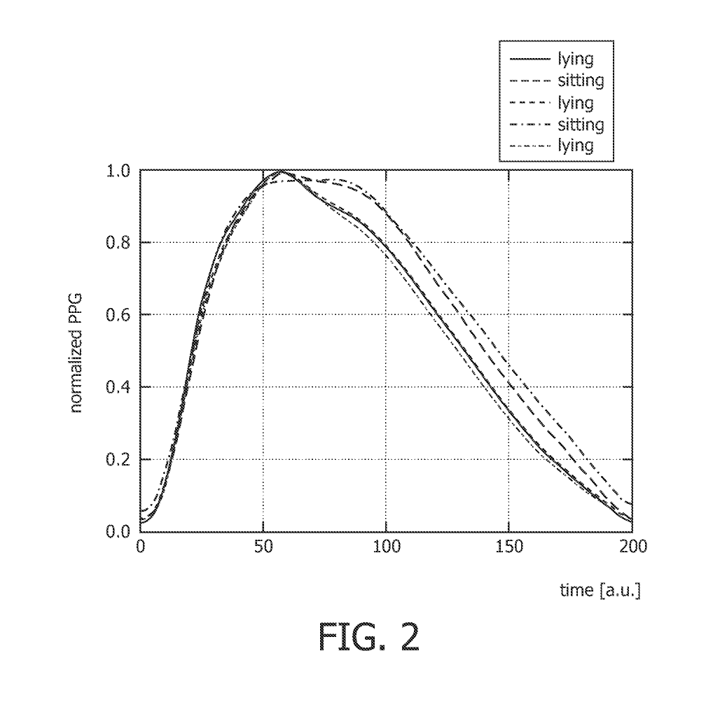

For example, FIG. 2 shows normalized PPG waveforms, extracted from a PPG signal as a function of time, taken from the ear of a single subject for a sequence of posture changes from lying to sitting exhibiting significant morphology changes of the PPG waveform. The x-axis represents a scaled time and the y-axis represents the normalized PPG signal. As is clearly visible, the PPG waveforms acquired for lying postures differ significantly from those acquired for sitting postures. However, the different PPG waveforms acquired for lying postures also differ mutually, which is also the case for the PPG waves acquired for sitting postures. Therefore, a reliable and routinely interpretation and analysis of PPG waveform morphologies related to a clinical context for this type of representation of the PGG signal, i.e. PPG signal as a function of time, is not possible from a conventional PPG diagram in which the PPG signal as a function of time is used for an analysis.

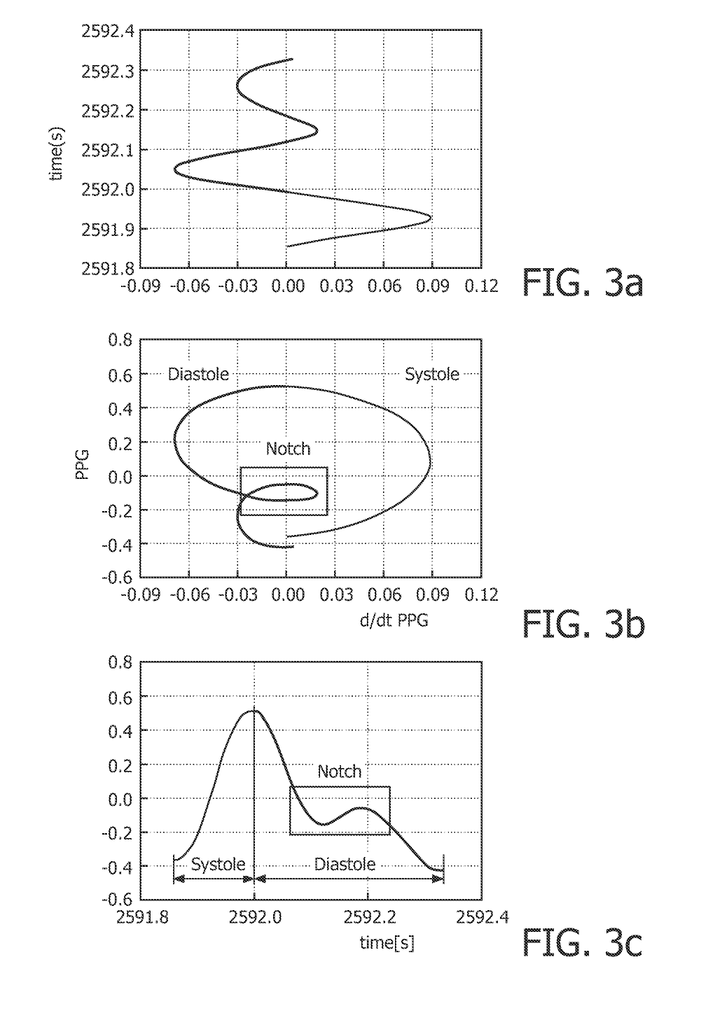

A basic concept of the invention is shown in FIG. 3. Diagram a) of FIG. 3 depicts a conventional x-y diagram of the PPG signal, wherein the x-axis represents the PPG signal and the y-axis represents the time. Diagram c) of FIG. 3 depicts an x-y diagram wherein the x-axis represents the time and the y-axis represents the derivative of the PPG signal with respect to the time, dPPG(t)/dt. The final result is shown in x-y diagram b) of FIG. 3 in which the x-axis represents the derivative of the PPG of interest with respect to time, dPPG (t)/dt, and the y-axis represents the PPG(t) signal. As one can recognize, the systolic and diastolic phases in diagram b) of FIG. 3 can easily be discriminated since the zero-crossings of the time derivative of the PPG signal mark the beginning of the systole, minimum of PPG in a heart cycle, and end of the systole, maximum of PPG in a heart cycle. In diagram b) of FIG. 3, the maximum amplitude of the PPG signal, the maximum slope of the PPG signal in systole and minimum slope of the PPG signal in diastole and the dicrotic notch of the PPG signal can be clearly recognized in diagram b) of FIG. 3 by, respectively, the maximum value of PPG, the minimum value of dPPG(t)/dt or the left extreme of the big loop, the maximum value of dPPG(t)/dt or the right extreme of the big loop, and the small inner loop. Alternatively, instead of a visual analysis of this diagram, an automatic analysis of the derivative of the PPG signal as a function of the PPG signal can be performed, wherein, for example, parameters are calculated that are representative of certain parts of the PPG waveform, such as maximum, minimum or extreme values of dPPG(t)/dt as a function of PPG(t) or the area of the small loop that characterizes the dicrotic notch. In this way the analysis of the derivative of the PPG signal with respect to time, dPPG (t)/dt, as a function of the PPG(t) signal provides for an easier recognition of PPG waveform patterns.

It should be noted that for all x-y diagrams the parameter represented by the x-axis and the parameter represented by the y-axis can also be exchanged. Furthermore, the analysis of the derivative of the PPG signal with respect to time as a function of the PPG signal may also be replaced by the vice versa situation, i.e. an analysis of the PPG signal as a function the derivative of the PPG(t) signal with respect to time.

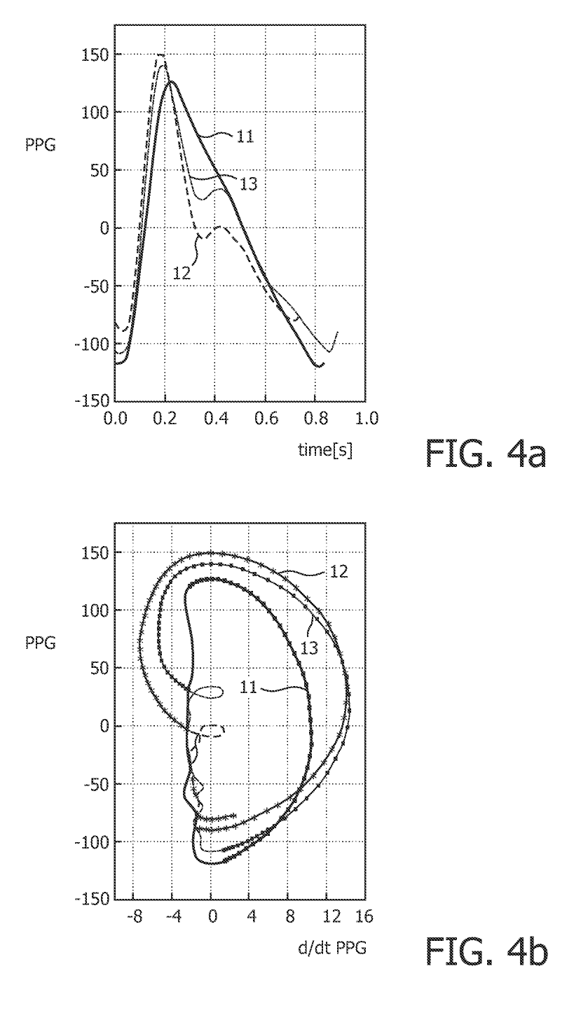

In the diagram a) on the left side of FIG. 4 three PPG signals 11, 12, 13 are displayed. The first PPG signal 11 is an initial measurement, the second PPG signal 12 is measured 4 minutes after Nitro administration, and the third PPG signal 13 is measured shortly before a faint. In the diagram b) on the right side of FIG. 4, the three PPG signals 11, 12, 13 are represented in an x-y diagram according to an embodiment of the invention. The x-axis represents the time derivative of PPG signal and the y-axis represents the PPG signal itself. The interpretation of the significant pulse shape changes is straightforward for the diagram b) on the right side of FIG. 4: a slope increase during systole for the second PPG signal 12 and the third PPG signal 13 with respect to the first PPG signal 11, a comparable pulse amplitude (difference between maximum and minimum value of the PPG signal), and almost no dicrotic notch for the first PPG signal 11 (no small inner loop), but a fully developed dicrotic notch for the second and third PPG signal 12, 13 as characterized by the small loops or straps.

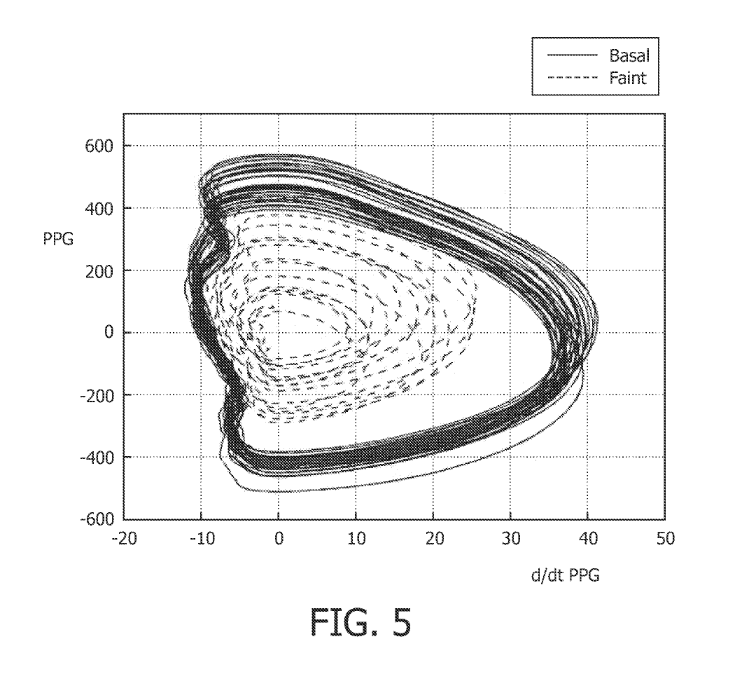

FIG. 5 shows the PPG signal in an x-y diagram according to the invention for a time period of about 1 minute at the beginning of a HUTT test and close to the manifestation of a faint, in which an oscillating PPG amplitude can be observed. Consequently, the appearance of an oscillating PPG graph in the x-y diagram is an easy to interpret signal pattern related to a significant change in the cardio-vascular state of the patient. In an embodiment according to the invention, the appearance of such patterns can be recognized by an automatic routine in a PPG measurement apparatus, such as in a pulse oximeter. When monitoring a patient, this allows for automatically issuing an alarm signal based on the output of the automatic analysis of dPPG(t)/dt as a function of PPG(t), for example to a central monitoring system.

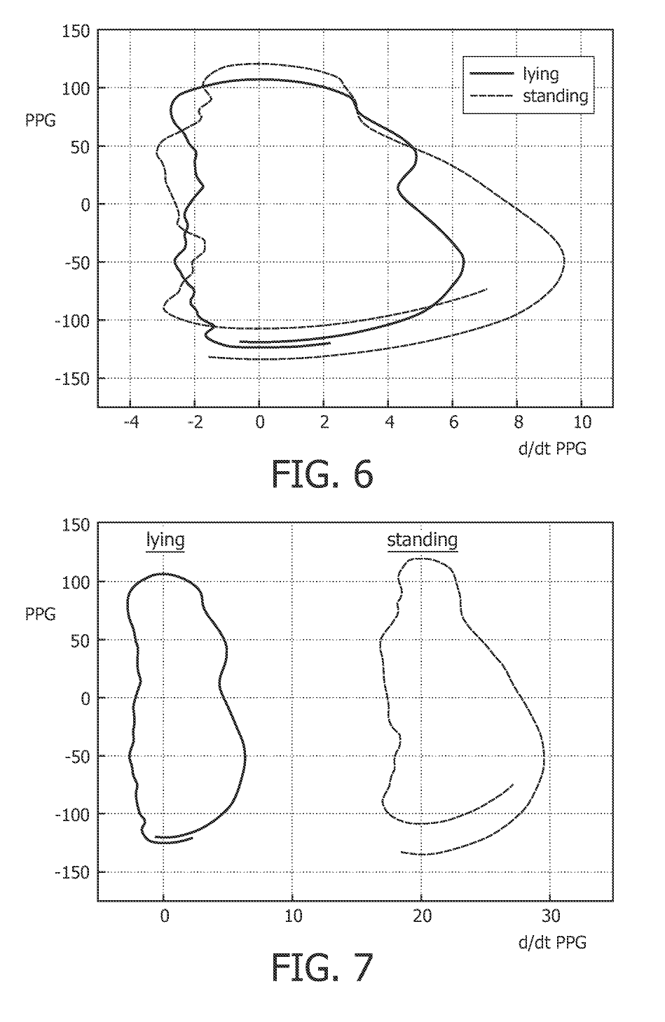

An alternative presentation of the signal can be provided by adding the variance of the PPG signals, for example represented by error bars, where the variance is derived from PPG measurements over a predefined time period. As mentioned before, the morphology of PPG waveforms depends on the state of the patient and on the specific measurement conditions when extracting the PPG signal, like for example the posture change of the patient, the physical activity of the patient, and the hydrostatic effect, for example in the case of a raised arm. Information on such conditions can be used as additional information for the analysis and interpretation of waveforms occurring in the PPG signal processing. One example is the change of the posture of the patient, which has significant impact on the morphology of the PPG waveform since the cardio-vascular regulation system compensates for gravitational effects like a reduced venous return in a standing position or posture of the patient compared to a lying position or posture of the patient. This is exemplified by FIG. 6 in which significant differences of the dPPG(t)/dt versus PPG(t) graph appear both in the systole phase and in the diastole phase as a function of the posture of the patient, in this case lying or standing.

To provide a more careful interpretation of the PPG signal, it is proposed in an embodiment according to the invention to separate PPG curves automatically depending on the measurement condition, for example depending on changes of the subject's posture. As information source for an automatic separation of the PPG graphs, a signal of a sensor detecting the posture of the subject can be used, like for example a signal of an acceleration sensor (ACC). If the respective signal is received from the sensor that detects a change in the posture of the subject, then, for example, an offset is set for the x-axis by adding a constant value to this part of the dPPG(t)/dt signal, thereby separating the dPPG(t)/dt versus PPG(t) graph measured at a different posture of the subject from the dPPG(t)/dt versus PPG(t) graph measured at the previous posture of the subject, in order to separate the dPPG(t)/dt versus PPG(t) graphs measured at different postures in the x-y diagram. FIG. 7 shows an example of this method where two dPPG(t)/dt versus PPG(t) graphs, that were acquired in a lying and a standing posture, are separated by adding a predefined offset to the derivative of the PPG, dPPG(t)/dt, that is acquired for the standing posture.

To make the interpretation of the PPG signal more robust, confidence intervals based on statistical data may be added to the analysis results and to the graphs. This will assist the physicians in distinguishing significant versus insignificant changes in the PPG signal. This may be implemented in the x-y diagram, for example by highlighting relevant areas of the diagram. To further assist the physician in the analysis of the PPG signal, the actual dPPG(t)/dt versus PPG(t) representation and/or specific characteristic parameters extracted there from, such as the dicrotic notch, is compared with dPPG(t)/dt versus PPG(t) graphs and extracted parameters that are related to a specific physiological condition. These specific dPPG(t)/dt versus PPG(t) graphs may be presented in the background of the actual PPG or in a separate area of a display unit.

In a further embodiment of the invention, the dPPG(t)/dt versus PPG(t) representation and/or the parameters extracted there from, is compared with PPG data that are retrieved by, for example, a statistical investigation of several subjects and which are stored in a storage medium of the PPG system. Such a comparison may be implemented in the system by, for example, a common comparison algorithm. If a significant overlap of the actual PPG with the stored PPG data is detected, the system can make a proposal to a physician for a physiological state of the patient based on the comparison with the statistical PPG data.

It should be understood that the proposed method can be realized by a computer program running on a computer system. The computer system may be equipped with an appropriate interface to receive data from a sensor capable of determining a property of blood in a tissue of a subject or patient.

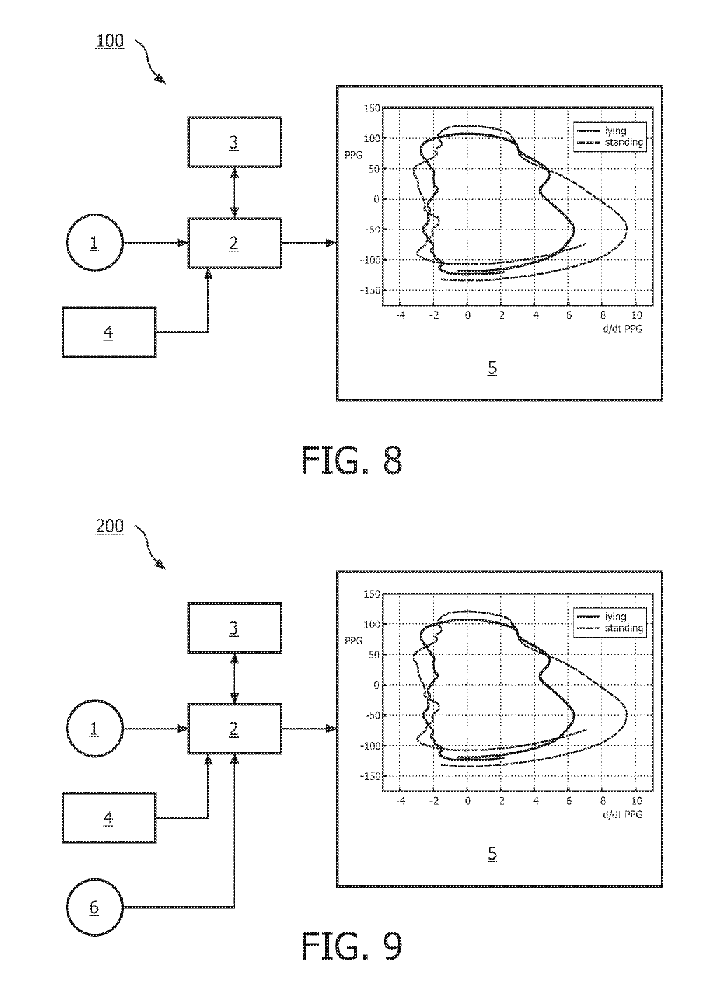

As stated before, according to a further aspect the invention relates to a photoplethysmograph measurement apparatus capable of processing a PPG signal. In FIG. 8 a schematic plot of a photoplethysmograph measurement apparatus 100 according to the invention is shown. Such a photoplethysmograph measurement apparatus 100, which may be, for example, part of a pulse oximeter, comprises a PPG sensor 1, a processor 2 and, in this embodiment, a display unit 5. The PPG sensor 1 capable of determining a property of blood of a patient, such as for example the relative amount of blood in a tissue of a patient, is connected to the processor 2 acting as processor of a PPG signal received from the PPG sensor 1. The processor 2 is connected to the display unit 5, a data storage device 3 and a user interface 4. While the data that is processed by the processor 2 is visualized by the display unit 5, the data storage device 3 is adapted to store the processed data for analysis at another time, for example for using the processed data as reference data. The user interface 4 is used to control the photoplethysmograph measurement apparatus 100. The processor 2 is adapted to calculate a derivative with respect to time of the PPG signal received from the sensor 1 and analyzes this derivative of the PPG signal with respect to time as a function of the PPG signal itself The PPG signal received from the sensor 1 is displayed on the display unit 5 on a second axis of an x-y diagram, for example the y-axis, and the derivative of the PPG signal calculated by the processor is displayed on a first axis of said x-y diagram, for example the x-axis. The display unit 5 may also display the results of the analysis of the derivative of the PPG signal as a function of the PPG signal in the form of parameters, for example by displaying characteristic features of this function in the form of parameters, for example the dicrotic notch. With the user interface 4 a physician can choose the most appropriate pre-processing steps of the PPG signals for the specific needs of a patient in a certain clinical context.

The derivative calculated by the processor 2 may be a first derivative of the

PPG signal with respect to the time or a higher derivative. The calculation of such derivatives can be implemented on the photoplethysmograph measurement apparatus 100 by a software and/or program code running on the processor.

In an embodiment of the invention, the photoplethysmograph measurement apparatus 100 is adapted to automatically compare the actual PPG signal with PPG signal data that are retrieved by, for example, a statistical investigation of several subjects and which are stored in the memory device 3 of the photoplethysmograph measurement apparatus 100, wherein both PPG data are represented as dPPG(t)/dt versus PPG(t). Such a comparison may be implemented in the apparatus by, for example, a common comparison algorithm that is implemented in the processor 2. If a significant overlap of the actual PPG data with the stored statistical PPG data is detected, the apparatus can provide for a proposal for a physiological condition of the patient or, alternatively, a proposal of a list of possible physiological conditions based on the comparison with the stored statistical PPG data.

In an embodiment, the appearance of specific patterns of the dPPG(t)/dt versus PPG(t) representation is recognized by an automatic routine in the processor 2. For example, the inner small loop in a dPPG(t)/dt versus PPG(t) diagram represents a dicrotic notch. When monitoring a patient, this allows for automatically issuing an alarm signal based on the output of the automatic routine of the processor, for example to a central monitoring unit.

In FIG. 9, a schematic plot of a further photoplethysmograph measurement 200 apparatus according to the invention is depicted. In general, the scheme corresponds to the scheme shown in FIG. 8, but the photoplethysmograph measurement apparatus 200 additionally comprises a posture sensor 6, like for example an acceleration (ACC) sensor. The posture sensor 6 is connected to the processor 2 and is capable of transmitting a signal to the processor 3 that is related to and depends on the posture of the monitored subject. These posture data can be taken into account by the processor 3 when analyzing the PPG signal, which is represented in the form of dPPG(t)/dt versus PPG(t), and/or when generating the visualization data of the PPG signal for displaying these data on the display unit 5 as described before.

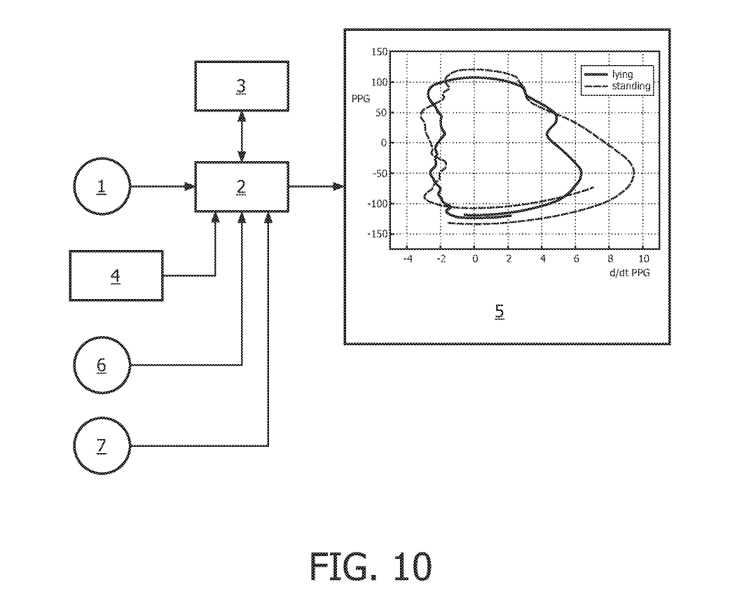

According to the schematic plot of FIG. 10 a further photoplethysmograph measurement apparatus 300 additionally comprises a second sensor 7, like for example the sensor of an ECG system or of a system to monitor the breathing activity of a patient, thereby providing additional data which are input to the processor 2. These additional data can be taken into account by the processor 2 analyzing the PPG signal, which is represented as dPPG(t)/dt versus PPG(t), and/or in generating the display data for displaying the PPG signal. Since sensors, like for example ECG sensors, are commonly integrated into patient monitoring systems, these sensors can also be used when integrating the inventive photoplethysmograph measurement apparatus 300 into a patient monitoring system. In an embodiment according to the invention, the photoplethysmograph measurement apparatus 300 is triggered by the data provided by the second sensor 7. For example, the photoplethysmograph measurement apparatus 300 can start to record a PPG signal, for example after the evolution of the QRS-complex. Therefore, the second sensor signal can be used to gate or trigger the PPG signal. Also a correlation of the PPG signal with the data provided by the second sensor 7 is possible which further improve the robustness and accuracy of the analysis and interpretation of the PPG signal.

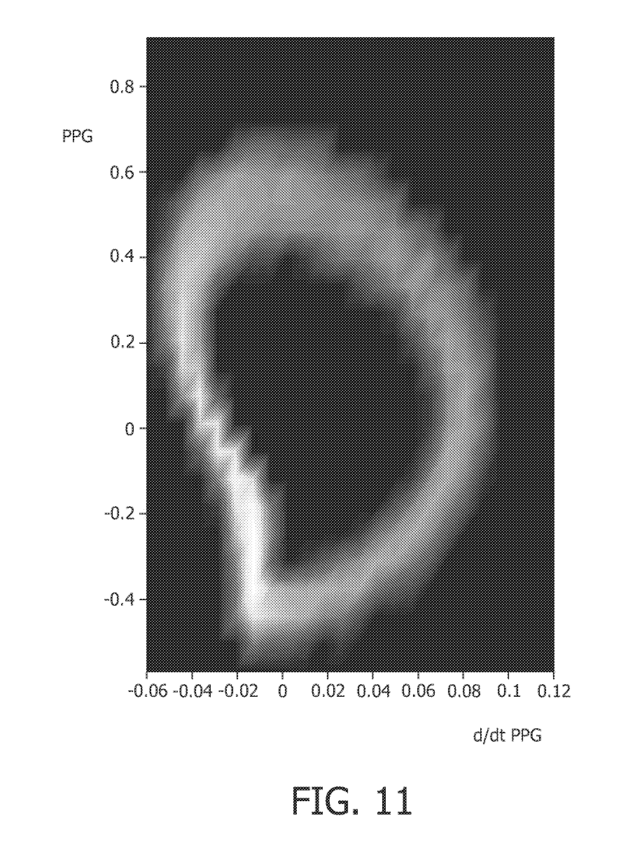

In FIG. 11, a dPPG(t)/dt versus PPG(t) representation of a PPG signal according to an embodiment of the invention is shown. The PPG signal as shown is recorded over a time period of 1 minute. The recorded and displayed PPG can be used as basal or initial information about the cardio-vascular state of a patient. A change of the cardio-vascular state of the patient will cause a difference between the actual dPPG(t)/dt versus PPG(t) representation and the basal or initial dPPG(t)/dt versus PPG(t) representation. The analyzed and reported difference can be used by a physician to interpret the cardio-vascular state of the patient.

It should also be understood that the proposed apparatus 100, 200, 300 can be part of a patient monitor.

In summary, the invention relates to the field of photoplethysmography, and in particular relates to a method of and apparatus for processing photoplethysmograph signals to support the analysis of photoplethysmograph signals in clinical scenarios. A derivative of a photoplethysmograph signal acquired over a time period is calculated. The derivative of the acquired photoplethysmograph signal with respect to time is analyzed as a function of the acquired photoplethysmograph signal or vice versa.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A method of processing a photoplethysmograph signal retrieved from a subject, said method comprising the steps of:
acquiring the photoplethysmograph signal over a time period;
calculating a derivative with respect to time of the acquired photoplethysmograph signal; and
analyzing the derivative of the acquired photoplethysmograph signal as a function of the acquired photoplethysmograph signal or vice versa.

2. The method according to claim 1, wherein the derivative of the photoplethysmograph signal is a first derivative of the acquired photoplethysmograph signal with respect to time.

3. The method according to claim 1, wherein the step of analyzing comprises a step of comparing the derivative of the acquired photoplethysmograph signal as a function of the acquired photoplethysmograph signal with the derivative of a second photoplethysmograph signal as a function of a second photoplethysmograph signal, which second photoplethysmograph signal represents a specific physiological condition.

4. The method according to claim 1, wherein the acquired photoplethysmograph signal is displayed in an x-y diagram, wherein a first axis of the x-y diagram represents the derivative of the acquired photoplethysmograph signal, and a second axis of the x-y diagram represents the acquired photoplethysmograph signal.

5. The method according to claim 4, wherein photoplethysmograph signals acquired during different time periods are displayed in one x-y diagram.

6. The method according to claim 4, wherein at least two photoplethysmograph signals, which are acquired at different time periods, are displayed in the x-y diagram with an offset on the first axis with respect to each other.

7. The method according to claim 6, further comprising the step of monitoring a posture of the subject and wherein the offset is induced by a change of the posture of the subject.

8. A photoplethysmograph measurement apparatus comprising:
a sensor for acquiring a photoplethysmograph signal over a time period corresponding to a property of blood in the subject tissue, and
a processor connected to the sensor and adapted to receive and process the photoplethysmograph signal from the sensor,
wherein the processor is adapted to calculate a derivative with respect to time of the photoplethysmograph signal received from the sensor, and to analyze the derivative of the photoplethysmograph signal as a function of the photoplethysmograph signal or vice versa.

9. The photoplethysmograph measurement apparatus according to claim 8, wherein the processor is adapted to extract a parameter that characterizes at least a part of the x-y diagram.

10. The photoplethysmograph measurement apparatus according to claim 8, further comprising a display unit connected to the processor for displaying an x-y diagram, wherein a first axis of the x-y diagram on the display unit represents the derivative of the acquired photoplethysmograph signal, and a second axis of the x-y diagram represents the photoplethysmograph signal.

11. The photoplethysmograph measurement apparatus according to claim 8, wherein the processor calculates the first derivative with respect to the time of the photoplethysmograph signal received from the sensor.

12. The photoplethysmograph measurement apparatus according to claim 8, further comprising a posture sensor indicating the posture of the subject monitored by the photoplethysmograph measurement apparatus and wherein the processor is adapted to receive and process signals from the posture sensor.

13. A patient monitoring system comprising the photoplethysmograph measurement apparatus according to claim 8.

14. A computer program for instructing a computer to perform the method according to claim 1.

15. A computer-readable medium such as a storage device, such as a floppy disk, CD, DVD, Blue Ray disk, or a random access memory (RAM), containing a set of instructions that causes a computer to perform a method according to claim 1.
